(19) United States
(12) Patent Application Publication
(10) Pub. No.: US 2013/0190806 A1
(43) Pub. Date: Jul. 25, 2013

(54) Tourniquet Apparatus for Controlling Blood Penetration (US NP)

(71) Applicants: James A. McEwen, Vancouver (CA); Michael Jameson, North Vancouver (CA); William K.W. Cheung, Vancouver (CA); Michael A. Gebert, New Westminister (CA)

(72) Inventors: James A. McEwen, Vancouver (CA); Michael Jameson, North Vancouver (CA); William K.W. Cheung, Vancouver (CA); Michael A. Gebert, New Westminister (CA)

(73) Assignee: WESTERN CLINICAL ENGINEERING LTD., Vancouver, BC (CA)

(21) Appl. No.: 13/574,413

(22) PCT Filed: Jan. 22, 2010
(86) PCT No.: PCT/CA10/00106

§ 371 Date: Feb. 4, 2013

(51) Int. Cl. A61B 17/135

(52) U.S. Cl. CPC A61B 17/1355
USPC 606/202

(57) ABSTRACT

An apparatus for estimating the distance of penetration (66) of arterial blood into a portion of a patient limb (6) encircled by a tourniquet cuff (2) comprising a cuff (2), a physiologic transducer (24), estimation means (74) and control means (70). The physiologic transducer (24) is associated with the cuff (2) and adapted for sensing a physiologic parameter indicative of penetration of arterial blood into the limb portion (6) encircled by the cuff (2) while blood flow past the limb portion (6) is stopped. The estimation means (74) responds to an output of the physiologic transducer (24) and produces an estimate of penetration of arterial blood past the proximal edge (60) of the cuff (2). The control means (70) responds to the estimation means (74) for facilitating the control of the pressure applied to the patient limb (6) by the cuff (2) to stop the flow of blood in the artery (58) by maintaining the estimated distance of penetration (66) near a selected penetration distance.

### FIELD OF THE INVENTION

This invention pertains to tourniquet systems commonly used for stopping the flow of arterial blood past a tourniquet cuff applied to a surgical patient's limb to facilitate the performance of a surgical procedure.

### BACKGROUND OF THE INVENTION

Typical surgical tourniquet systems of the prior art include a tourniquet cuff which encircles the limb of a surgical patient and a tourniquet instrument which is releasably connected to an inflatable portion within the tourniquet cuff through a length of tubing, thereby establishing a gas-tight passageway between the cuff and the tourniquet instrument. The tourniquet instrument supplies pressurized gas to inflate and regulate the pressure in the tourniquet cuff above a minimum pressure required to stop arterial blood flow distal to the cuff, for a duration suitably long for the performance of a surgical procedure. Many types of surgical tourniquet systems have been described in the prior art, such as those described by McEwen in U.S. Pat. No. 4,469,099, No. 4,479,494, No. 5,439,477 and McEwen and Jameson in U.S. Pat. No. 5,556,415 and No. 5,855,589.

Studies published in the surgical literature have shown that the safest tourniquet pressure is the lowest pressure that will stop the flow of arterial blood past a specific cuff applied to a specific patient for the duration of that patient's surgery. Such studies have shown that higher tourniquet pressures are associated with higher risks of tourniquet-related injuries to the patient. Therefore, when a tourniquet is used in surgery, surgical staff generally try to use the lowest tourniquet pressure that in their judgment is safely possible.

The inward compressive force applied to a limb by a pressurized tourniquet cuff to close underlying arteries is not equal across the width of the cuff, from proximal to distal edges. Consequently when inflated to a minimum pressure required to stop arterial blood flow past the distal edge of the tourniquet cuff, arterial blood still penetrates beneath the proximal edge of the cuff for some distance to a location where the arteries become closed. In addition to the pneumatic pressure to which a selected tourniquet cuff is inflated, several variables affect the distance to which arterial blood penetrates beneath the cuff. These variables include: the patient's limb characteristics (for example, limb shape, circumference and soft tissue characteristics at the cuff location); characteristics of the selected tourniquet cuff (for example, cuff design, cuff shape and cuff width); the technique of application of the cuff to the limb (for example, the degree of snugness or looseness of application and the absence, presence and type of underlying limb protection sleeve); physiologic characteristics of the patient including blood pressure and limb temperature; the anesthetic technique employed during surgery (for example, whether a general or regional anesthetic is given, the types and dosages of anesthetic agents employed and the degree of attention paid to anesthetic management); the length of time the tourniquet remains inflated on the limb; changes in limb position during surgery; and any shift in the location of the cuff relative to the limb during surgery.

In U.S. Pat. No. 6,605,103 Hovanes et al. describe apparatus for detecting the flow of blood past a tourniquet cuff and into a surgical field. Such prior-art apparatus is impractical because blood must flow past the tourniquet cuff before it can be detected, requiring surgical staff to do one of two things if blood enters the surgical field: interrupt the surgical procedure and take action to remove the blood; or proceed with blood in the field which might affect visualization and the quality of surgery. Further, Hovanes et al. relies on the accurate sensing of the onset of blood flow past a tourniquet cuff by the measurement of blood flow-related signals such as acoustic Korotkoff sounds; such apparatus can only be used when pulsatile arterial blood is actually flowing past the tourniquet cuff toward the surgical field, and can be difficult and inaccurate because sensing of onset of pulsatile blood flow past the cuff requires measurement of a very small signal in the presence of large levels of noise created by limb movement, pneumatic cuff pressure regulation, and changes in a range of physiologic variables.

An ultrasonic tourniquet system is described by McEwen et al. in PCT International Patent App. WO 2009/012594, hereby incorporated by reference. This system adapts ultrasonic Doppler techniques to sense the flow of arterial blood within a portion of a limb beneath an encircling tourniquet cuff Detection of arterial blood flow within a limb beneath a tourniquet cuff by adapting ultrasonic Doppler apparatus and methods requires the accurate measurement of small pulsatile signals in the presence of relatively large levels of noise, especially as the amount of arterial blood flowing beneath the cuff decreases. Further, detection of blood flow by the apparatus of McEwen et al. must be rapid as well as being accurate, to facilitate dynamic and accurate control of tourniquet pressure during surgery.

It is important for surgical patient safety that tourniquet cuffs have inflatable portions that completely encircle the limb when correctly applied, so that tourniquet pressure may be applied uniformly around the limb and thus minimize injuries to limb tissues. Some “cylindrical” tourniquet cuffs of the prior art have a rectangular shape and are ideally suited for application to patients with cylindrical limbs Other prior-art tourniquet cuffs have an arcuate shape, and such “contour cuffs” are better suited for patients having tapered limbs, allowing pressures to be transferred optimally to tissues of tapered limbs between proximal and distal cuff edges. Some tourniquet and non-tourniquet cuffs of the prior art are adapted for inclusion of physiologic transducers, but such adaptation may prevent or detrimentally affect the uniform application of pressure circumferentially around the limb, and may prevent or detrimentally affect the desired application of pressure between proximal and distal edges of the cuff. In U.S. Pat. No. 6,231,507 and U.S. Pat. No. 6,361,396, Zikorus et al. describe a cuff having an ultrasonic window to facilitate manual positioning of a separate ultrasonic sensor by an operator. The prior-art cuff of Zikorus is comprised of two regions along its length: a non-inflating region that includes a window for the ultrasonic sensor and a separate inflating region spaced apart from the window, so that the inflating region encircles only a portion of an underlying limb when the cuff is applied. Without an inflatable portion that completely encircles the limb, prior-art cuff apparatus such as that of Zikorus et al. apply non-uniform pressures around the limb that may result in injuries to nerves, muscles and other soft tissues, especially if the cuff is pressurized to levels sufficiently high to stop blood flow for periods of time that are suitably long to carry out surgical procedures.

There is a need for tourniquet apparatus that can accurately and reliably apply pressure to a limb or to a selected blood vessel to stop blood flow, and that can accurately and reliably measure the distance of penetration of arterial blood beneath the pressure-applying apparatus while blood flow past the apparatus is stopped. There is a further need for apparatus that can monitor and control the distance of penetration of blood past the proximal edge of a tourniquet cuff when blood flow past the cuff is stopped, thereby facilitating improvements in tourniquet safety during surgery and in other settings.

### BRIEF DESCRIPTION OF THE DRAWINGS

FIG. 1 is a pictorial representation of the preferred embodiment in a surgical application.

FIG. 2 is a view of the contour cuff of the preferred embodiment laid flat.

FIG. 3 is a view of the cylindrical cuff of the preferred embodiment laid flat.

FIG. 4 is a view of the cylindrical cuff of the preferred embodiment applied to a patient limb.

FIG. 5 is a view of the contour cuff of the preferred embodiment applied to a patient limb.

FIG. 6A depicts an artery under the cuff while blood flow past the cuff is not stopped.

FIG. 6B depicts an artery under the cuff while blood flow past the cuff is stopped.

FIG. 6C depicts an artery under the cuff while blood flow past the cuff is stopped.

FIG. 7 is a block diagram of the preferred embodiment.

### DETAILED DESCRIPTION OF THE PREFERRED EMBODIMENT

A specific embodiment illustrated is not intended to be exhaustive or to limit the invention to the precise form disclosed. It is chosen and described in order to explain the principles of the invention and its application and practical use, and thereby enable others skilled in the art to utilize the invention.

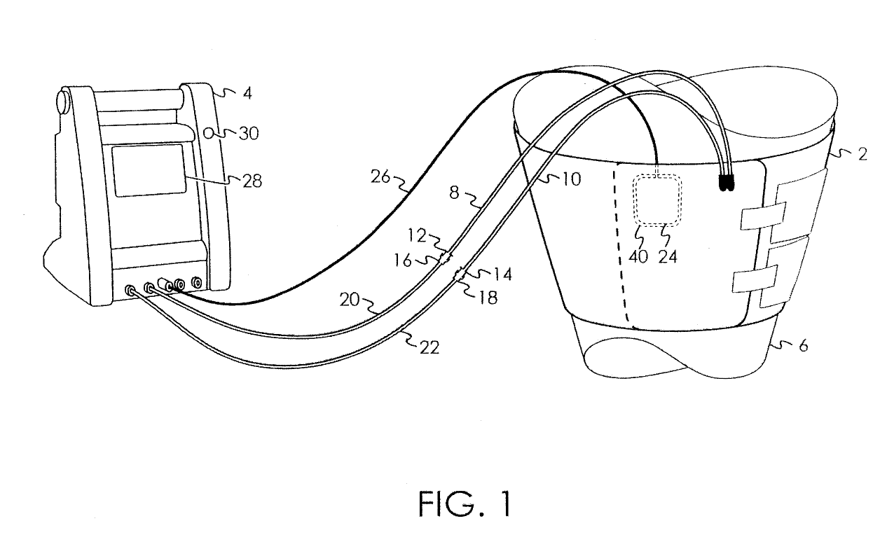

FIG. 1 depicts the preferred embodiment in a surgical application. Contour tourniquet cuff 2 is shown pneumatically connected to instrument 4 and secured around a portion of tapered patient limb 6. Cuff 2 includes an inflatable portion that is substantially the same length and width as contour cuff 2. The length of the inflatable portion of cuff 2 is sufficient for the inflatable portion to overlap upon itself and form a gas passageway that completely surrounds the portion of limb 6 to which cuff 2 has been secured. When the inflatable portion of cuff 2 is filled with pressurized gas from instrument 4 inward compression is applied to limb 6. Pressure applied by cuff 2 to limb 6 acts to close arteries within the limb beneath cuff 2 and prevent arterial blood from flowing past the distal edge of cuff 2.

The inflatable portion of cuff 2 is pneumatically connected to instrument 4 by two gas passageways. Separate pneumatic passageways to the inflatable portion of cuff 2 are provided by cuff port 8 and cuff port 10. As shown in FIG. 1 cuff port 8 and cuff port 10 are of sufficient length to allow pneumatic connections to cuff 2 to be made outside of a sterile surgical field. Cuff port 8 and 10 are fitted with male locking connectors 12 and 14 (DSM2202, Colder Products Company, St. Paul, Minn.) respectively, and mate to form releasable pneumatic connections with female locking connectors 16 and 18 (PMC1704, Colder Products Company, St. Paul, Minn.). The connectors illustrated in FIG. 1 are shown connected and form part of the pneumatic passageways between instrument 4 and cuff 2. Pneumatic connections from instrument 4 to cuff 2 are made by flexible plastic tubing 20 and 22 which are fitted with female locking connectors 16 and 18 respectively.

Physiologic transducer 24 adjoins the inflatable portion of cuff 2 to accurately and reliably sense a physiologic parameter indicative of the distance of penetration of arterial blood past the proximal edge of cuff 2 while blood flow past the distal edge of cuff 2 is stopped. In the preferred embodiment physiologic transducer 24 is an ultrasound transducer adapted to quantify the diameter of arterial blood vessels as described further below. The physiologic transducer 24 may use other technologies either alone or in combination for detecting blood penetration, including optical technologies, electrical conductivity technologies, and adaptations of tonography. For example, to detect blood penetration by adapting a tonometer, pressure fluctuations at points along the surface of the limb caused by pulsatile variations in blood pressure in the underlying arteries are measured. The distance arterial blood penetrates can be estimated by the location beneath the cuff that pressure fluctuations cease to be detectable.

Physiologic transducer 24 is shown connected to instrument 4 via cable 26. Alternatively, physiologic transducer 24 could be configured to communicate wirelessly with instrument 4, eliminating the need for cable 26.

Instrument 4 includes user interface 28 which is comprised of a touch sensitive graphic display panel. User interface 28 includes indicators and controls to enable a user to: inflate and deflate cuff 2; set a desired pressure to be maintained within the inflatable portion of cuff 2; set a desired distance of penetration of arterial blood to be maintained past the proximal edge of cuff 2; and other indicators and controls required for the operation of instrument 4.

Alarm indictor 30 shown in FIG. 1 is a bright red light emitting diode (LED) which is activated by instrument 4 in response to detected alarm conditions. Instrument 4 also signals the presence of an alarm condition by generating an audible tone to further alert the user to the presence of an alarm condition and displays alarm text messages describing the alarm condition via user interface 28. One example of a detected alarm condition that requires the user's attention is when the distance of penetration of arterial blood into the limb portion exceeds a predetermined distance threshold.

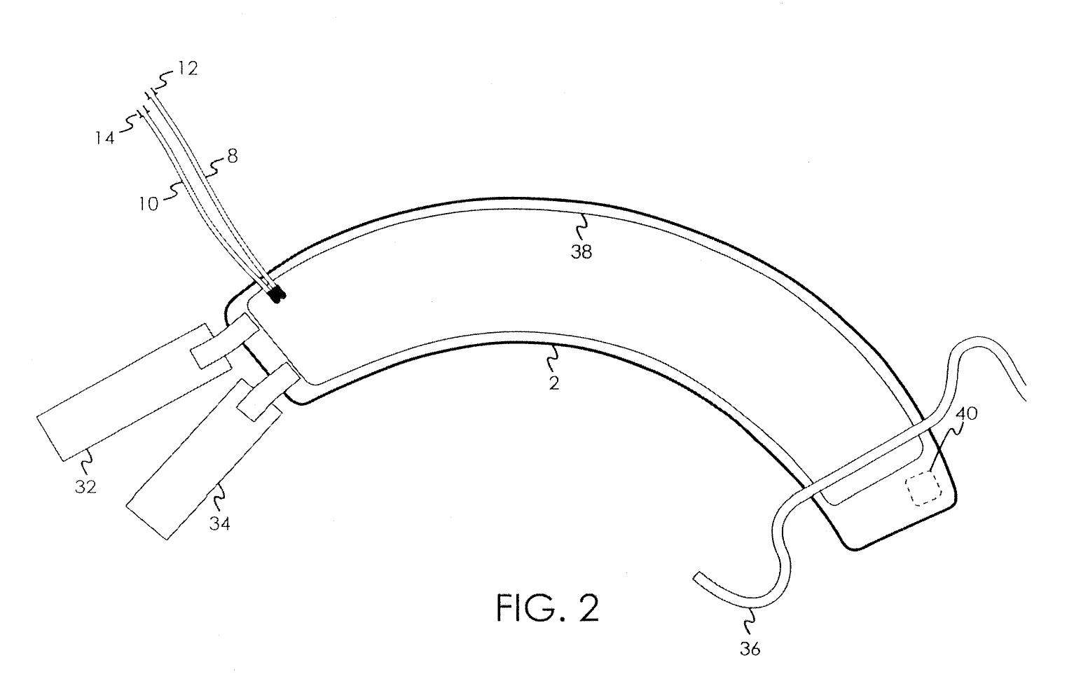

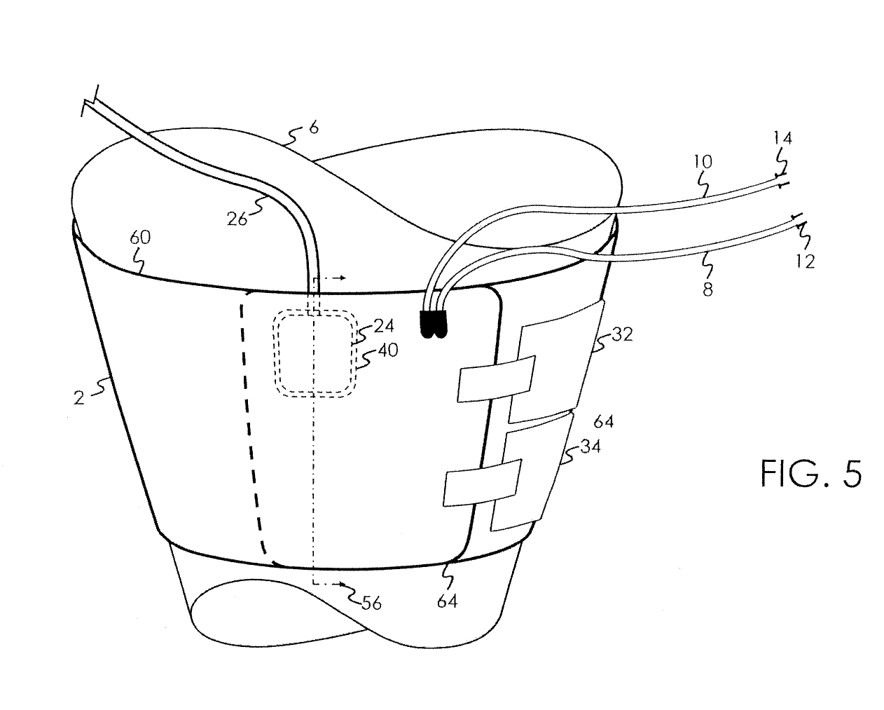

Contour cuff 2 shown in FIGS. 1, 2, 5 and 6 is similar in design and construction to the cuff described by McEwen et al. in U.S. Pat. Pub. No. 2007/0219580. As shown in the laid flat view in FIG. 2 contour cuff 2 has a substantially arcuate shape with the width of the cuff reduced near the end edges. The arcuate shape of cuff 2 and the degree to which the width near the end edges is reduced are predetermined to allow cuff 2 to be applied to limbs with a predetermined range of tapers such that contour cuff 2 remains substantially in contact with the limb along its width around the circumference of a patient limb as shown in FIGS. 1 and 5. The side edge of contour cuff 2 with the greater radius is the proximal side edge and the side edge with the lesser radius is the distal side edge when contour cuff 2 is correctly applied to a limb.

Contour cuff 2 is secured around the limb by securing straps 32 and 34. Securing straps 32 and 34 are non-releasably attached to a non-inflating region of contour cuff 2 near an end edge. Securing straps 32 and 34 have fastening portions which releasably engage with the outer surface of cuff 2 and bending portions which permit the fastening portions to be positioned such that they can completely engage the outer surface within the side edges of contour cuff 2. In the preferred embodiment the outer surface of contour cuff 2 and the fastening portions of securing straps 32 and 34 are formed from hook and loop materials. The outer surface of cuff 2 is a loop type material and the fastening portions of securing straps 32 and 34 are formed from hook type material. Tie strap 36, shown in FIG. 2, provides a means for the user to align and pull cuff 2 snug around limb 6. When contour cuff 2 has been secured around limb 6 the ends of tie strap 36 may be tied together to help maintain the overlapping portion of the cuff in alignment around limb 6 by preventing the cuff from twisting, telescoping and rolling on the limb when pressurized. For clarity, tie strap 36 is not shown in FIG. 5.

The inflatable portion of cuff 2 is bounded by bladder seal 38 as shown in FIG. 2.

Transducer region 40 shown in FIGS. 1, 2, 5 and 6 is a portion of cuff 2 configured to match the size and shape of physiologic transducer 24 and to retain physiologic transducer 24 in a fixed position relative to cuff 2. Transducer region 40 adjoins the inflatable portion of cuff 2 near the proximal side edge. This location is selected to permit physiologic transducer 24 to sense a physiologic parameter indicative of the distance of penetration of arterial blood past the proximal edge of cuff 2 and to maintain a substantially uniform distribution of pressure by cuff 2 to limb 6. Mating hook and loop fasteners are used to attach physiologic transducer 24 to the inner surface (the side closet to the limb) of cuff 2 at transducer region 40. The inner surface of cuff 2 at transducer region 40 is fitted with loop material and the back surface of physiologic transducer 40 is fitted with hook material. Alternatively other types of fastening methods known in the art may be used to retain physiologic transducer 24 in position at transducer region 40. The location of transducer region 40 at the end edge opposite to the end edge to which the securing straps are attached permits the inflatable portion of cuff 2 to overlap transducer region 40 when cuff 2 is correctly applied to a limb such that the inflating portion of the cuff overlaps upon itself. The inflatable portion of cuff 2 that overlaps at transducer region 40 helps to maintain a longitudinal axis of the surface of physiologic transducer 24 at a predetermined angle relative to the long axis of patient limb 6 and in contact with the surface of patient limb 6 and also helps insure uniform pressure application to limb 6 by cuff 2.

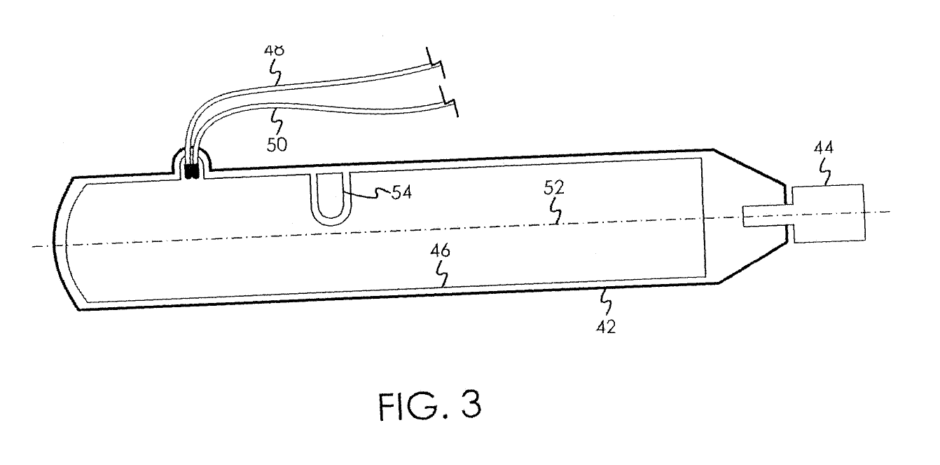

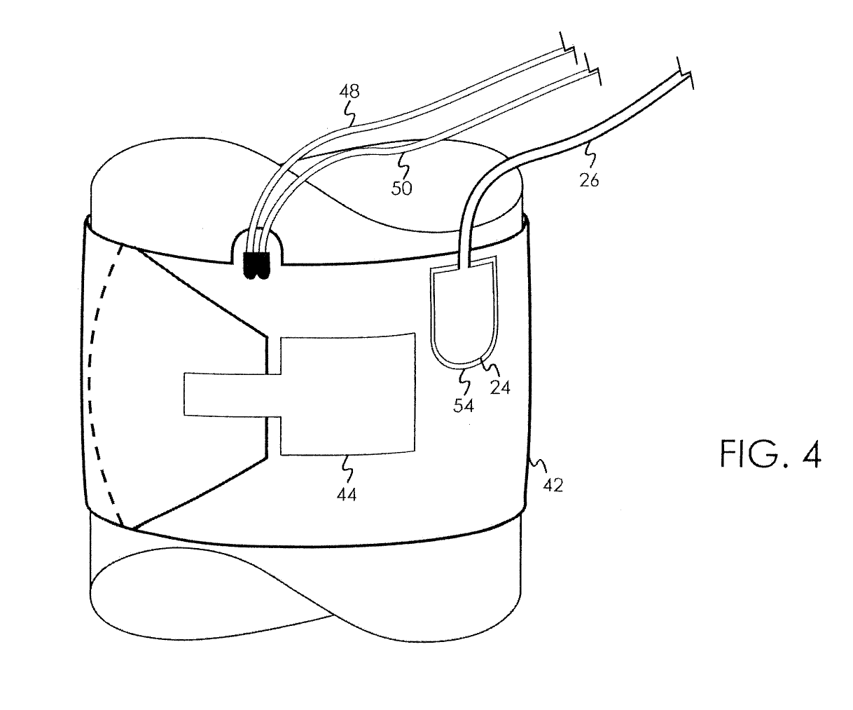

An alternate form of the cuff of the preferred embodiment is shown in FIGS. 3 and 4. Cylindrical cuff 42 is formed from polyurethane coated nylon fabric and is similar in design and construction to the cuff described by McEwen et al. in U.S. Pat. Pub. No. 2007/0244506. As shown in FIG. 3 cylindrical cuff 42 has a substantially rectangular shape designed to be applied on cylindrical shaped limbs as shown in FIG. 4.

Cylindrical cuff 42 is secured around the limb by securing strap 44. Securing strap 44 is non-releasably attached to a non-inflating region of cylindrical cuff 42 near an end edge. In the preferred embodiment the outer surface of cylindrical cuff 42 and the fastening portion of securing strap 44 are formed from hook and loop type materials. The outer surface of cuff 42 is a loop type material and the fastening portion of securing strap 44 are formed from hook type material.

The inflatable portion of cylindrical cuff 42 is bounded by bladder seal 46 as shown in FIG. 3. Cuff ports 48 and 50 shown in FIGS. 3 and 4 are attached to cuff 42 on the proximal side of cuff midline 52. Transducer region 54 adjoins the inflatable portion of cuff 42 on the proximal side of cuff midline 52. The location and properties of transducer region 54 are selected to isolate physiologic transducer 24 from the inflatable portion of cuff 42 and to permit cuff 42 to maintain a substantially uniform distribution of pressure to the limb. Physiologic transducer 24 is positioned on the outer surface (side away from the limb) of transducer region 54 and secured in place by retaining straps (not shown) such that the longitudinal axis of the surface of physiologic transducer 24 is maintained at a predetermined angle relative to the long axis of patient limb 6. In this configuration the material properties of cuff 42 at the location of transducer region 54 are selected to permit physiologic transducer 24 to be able sense the distance of penetration of arterial blood through cuff 42 at transducer region 54. In the preferred embodiment the material properties of cuff 42 at transducer region 54 are selected to permit the transmission of ultrasound and transducer region 54 is formed from flexible polyurethane film or alternatively high density polyethylene sheeting. It will be appreciated that if a different sensing technology is used by physiologic transducer 24 to detect the penetration of arterial blood, different material properties at transducer region 54 may be selected to match the sensing technology employed by physiologic 24. For example if physiologic transducer 24 employs optical technology to sense a parameter indicative of the distance of penetration of arterial blood, the materials of cuff 42 at transducer region 54 may be selected to be transparent to the wavelengths of light used by physiologic transducer 24.

In the preferred embodiment physiologic transducer 24 comprises one or more arrays of piezoelectric crystal elements or capacitance micromachined ultrasonic transducer (CMUT) cells or other materials and technologies known in the prior art to be suitable for transmitting and receiving high frequency acoustic energy, as generally described for example by Khuri-yakub et al. (“Next-Gen Ultrasound”, B. Khuri-yakub, O. Oralkan, M. Kupnik, IEEE Spectrum, 46:5, May 2009), hereby incorporated by reference.

. By adjusting the relative phases of electronic signals applied to the crystal elements that comprise an array the ultrasound waves produced by the array may be steered and focused to insonify a selected region within the portion of a patient limb beneath the physiologic transducer. The crystal elements of physiologic transducer 24 may be configured as a one-dimensional array or as a two-dimensional array. When a two dimensional array is used ultrasound waves produced by the array may be steered and focused to insonify a three-dimensional region within the limb beneath the transducer. It will be apparent that multiple transducers could be used to insonify a larger region of the portion of limb 6 encircled by contour cuff 2 or cylindrical cuff 42.

Instrument 4 operates physiologic transducer 24 to detect the distance arterial blood penetrates into the proximal portion of limb 6 encircled by contour cuff 2 or cylindrical cuff 42. Ultrasonic waves are emitted by physiologic transducer 24 at scanning angles relative to the surface of physiologic transducer 24 and traverse the tissue beneath physiologic transducer 24. The waves emitted by physiologic transducer 24 reflect off various tissue structures within the limb. Variations in the amplitude of the reflections allow different tissue structures to be identified, such as the walls of arteries. Doppler frequency shifts in the reflections indicate moving structures, such as the walls of arteries responding to blood pressure variations during cardiac cycles, and blood cells moving within arteries.

Physiologic transducer 24 operates to identify and locate arteries within the limb beneath the transducer that are inside the scanning region of the transducer. The lumen minimum diameters of the identified arteries are estimated at locations along their length in the scanning region of physiologic transducer 24. At the location along the length of an identified artery where the lumen diameter is estimated to be zero, the artery is closed.

Arterial blood can penetrate proximally into the portion of limb beneath cuff 2 to the location where the arteries carrying the blood become closed. The location of physiologic transducer 24 relative to the proximal edge of contour cuff 2 or cylindrical cuff 42 permits the distance that arterial blood penetrates past the proximal edge of contour cuff 2 or cylindrical cuff 42 to be estimated as described further below.

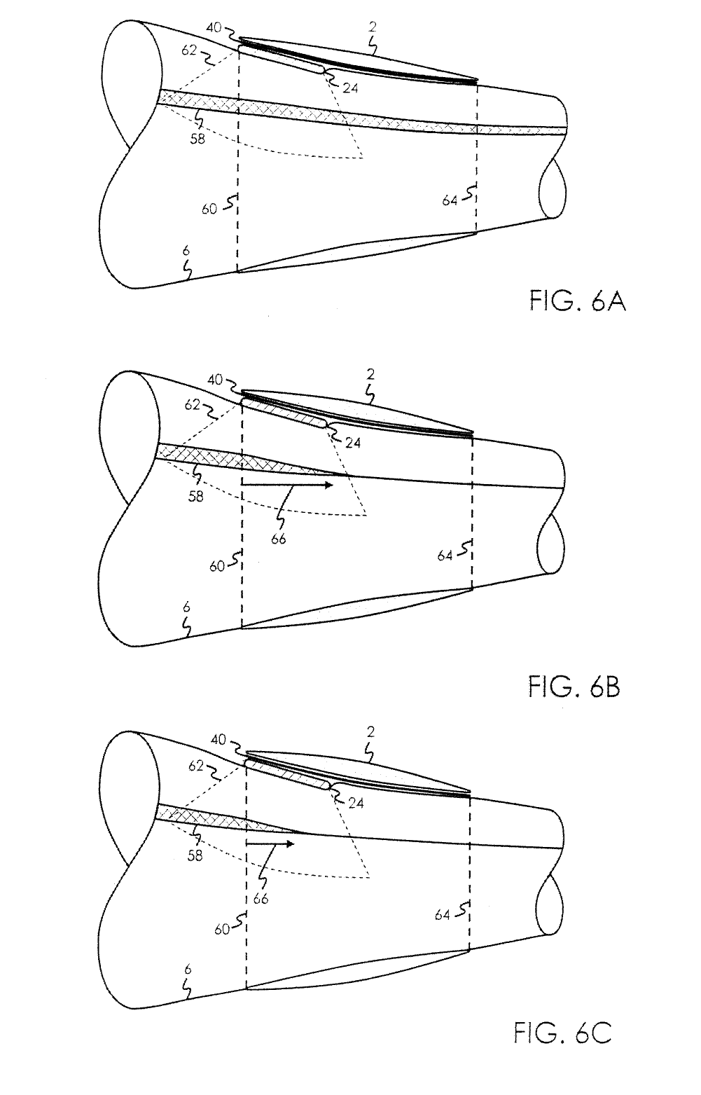

A more detailed view of contour cuff 2 applied to tapered patient limb 6 is shown in FIG. 5. FIGS. 6A, 6B and 6C are cross sectional views at location 56 of FIG. 5 that depict an artery 58 within the portion of patient limb 6 encircled by cuff 2 and illustrate the effect of pressure applied by cuff 2 to patient limb 6 on the distance of penetration of arterial blood past proximal cuff edge 60 of cuff 2.

As can be seen in FIGS. 6A, 6B and 6C the inflatable portion of cuff 2 overlaps physiologic transducer 24 which is attached to cuff 2 at transducer region 40. Sensing region 62 represents the volume of tissue of patient limb 6 insonified by physiologic transducer 24 in which arterial lumens can be characterized and the distance of penetration of arterial blood estimated.

In FIG. 6A the inflatable portion of cuff 2 is at a pressure that permits arterial blood to flow past distal cuff edge 64 of cuff 2. As can be seen in FIG. 6A, artery 58 is not closed and blood is free to flow past distal cuff edge 64.

FIGS. 6B and 6C illustrate the effect of an increase in the level of gas pressure within the inflatable portion of cuff 2 on the distance of penetration of arterial blood past proximal cuff edge 60 while blood flow past distal cuff edge 64 is stopped.

FIG. 6B depicts the inflatable portion of cuff 2 inflated to a pressure level that causes cuff 2 to apply sufficient inward compression to limb 6 to close artery 58 at a point beneath cuff 2 and stop arterial blood flow past the distal cuff edge 64. Physiologic transducer 24 determines the location relative to cuff 2 that the lumen of artery 58 is closed thereby allowing a distance of penetration of arterial blood 66 to be estimated. The distance of penetration of arterial blood varies with each cardiac cycle as blood pressure changes. The estimated distance of penetration of arterial blood is defined as: the greatest distance measured relative to proximal cuff edge 60 of cuff 2 that blood penetrates within the arterial vessels underlying cuff 2 within one cardiac cycle.

FIG. 6C depicts the inflatable portion of cuff 2 inflated to a pressure level greater to that illustrated in FIG. 6B. As can be seen from the figure, an increased portion of artery 58 is closed and the estimated distance of penetration 66 is less than that shown in FIG. 6B.

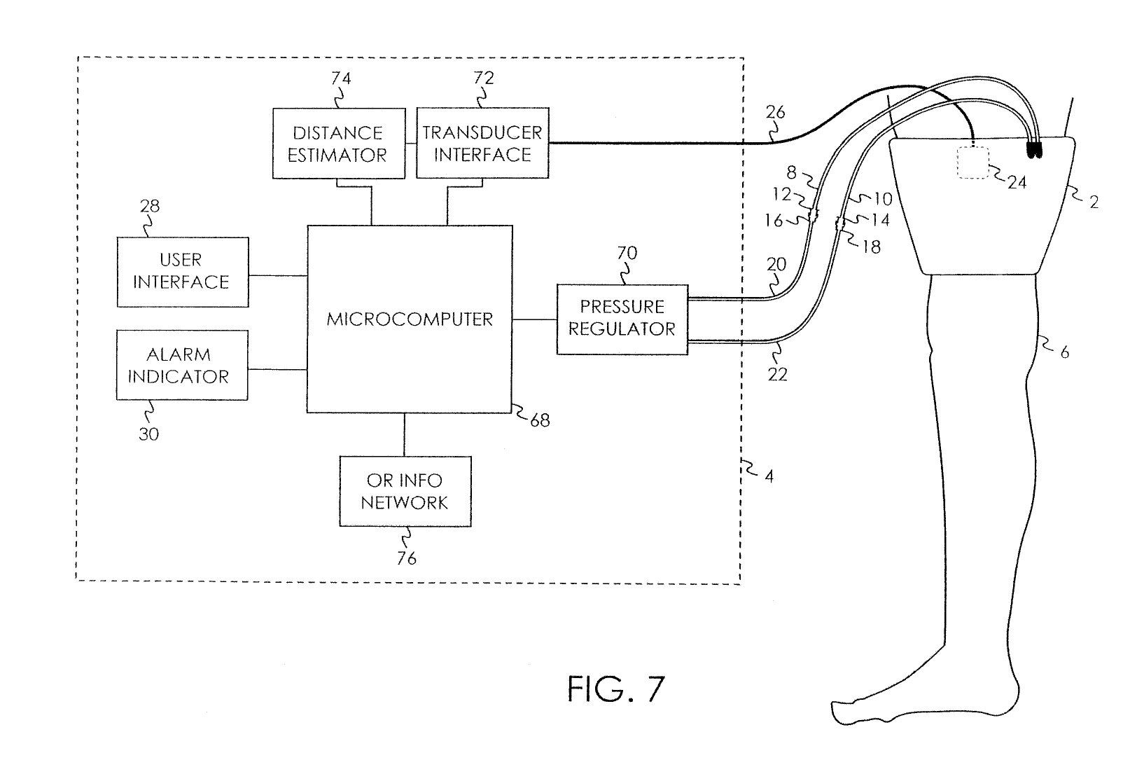

Referring to the block diagram of instrument 4 shown in FIG. 7, instrument 4 includes a microcomputer 68 with associated memory and control software, analog and digital peripheral interface circuitry, and other necessary support components for the operation of instrument 4.

Pressure regulator 70 of instrument 4 communicates pneumatically with the inflatable portion of cuff 2 and acts to regulate the pressure of gas within the inflatable portion at a level near a reference pressure level communicated to cuff pressure regulator 70 by microcomputer 68. Pressure regulator 70 also communicates the level of gas pressure within the inflatable portion of cuff 2 (cuff pressure level) to microcomputer 68. Although instrument 4 is shown and described with a single pressure regulator it will be apparent that additional pressure regulators could be included within instrument 4 to independently regulate the pressure in multiple cuffs to apply differing pressures to various selected portions of a limb.

Transducer interface 72 is the ultrasound engine of instrument 4 and includes transceivers for driving and receiving signals from the elements of physiologic transducer 24 and electronics for beam forming, steering, focusing, signal amplification, filtering, and signal processing functions. Transducer interface 72 acts to scan the volumes of tissue of limb 6 within the sensing region 62 of physiologic transducer 24 to identify arteries and determine the lumen diameter and cross sectional area of the identified arteries.

Transducer interface 72 communicates a parameter indicative of the distance of penetration of arterial blood to distance estimator 74. In the preferred embodiment the parameter communicated to distance estimator 74 is the cross sectional profile of along the length of the portion of an identified artery within the sensing region of physiologic transducer 24.

As described above distance estimator 74 receives a parameter indicative of the distance of penetration of arterial blood. In the preferred embodiment distance estimator 74 has stored in memory the relative geometric relationship between sensing region 62 of physiologic transducer 24 and the proximal cuff edge 60 of cuff 2. Alternatively, physiologic transducer 24 may be adapted to also sense its geometric orientation relative to the proximal cuff edge 60 of cuff 2 and communicate this information to distance estimator 74. Distance estimator 74 analyzes the cross sectional profile along the length of the portion of the identified artery within sensing region 62 during a cardiac cycle and determines the coordinates (depth and scan angle) within sensing region 62 of the locations relative to proximal cuff edge 60 at which the arterial lumen diameter is estimated to be zero. Using the known geometric relationship between sensing region 62, proximal cuff edge 60 and the determined coordinates within sensing region 62, distance estimator 74 calculates an estimate of the distance of penetration of blood beneath cuff 2 relative to proximal cuff edge 60. This is illustrated for identified artery 58 in FIGS. 6B and 6C as the location in the artery 58 adjacent to the head of arrow 66 (that is, the location nearest to proximal edge 60 that the artery is closed). For clarity, the operation of distance estimator has been described for a single identified artery; in the preferred embodiment distance estimator 72 estimates the distance of penetration of arterial blood in a plurality of identified arteries within sensing region 62 and determines the distance of penetration to be the greatest distance measured relative to proximal cuff edge 60 of cuff 2 that blood penetrates within the arterial vessels underlying cuff 2 within one cardiac cycle.

User interface 28 provides a means for a user to interact with instrument 4 as described above. Via user interface 28, a user may control the inflation and deflation of cuff 2, set cuff pressure reference levels and instruct instrument 4 to maintain the distance of penetration of arterial blood past the proximal edge of cuff 2 near a selected reference distance of penetration.

When instructed to do so, microcomputer 68 operates to control the distance of penetration of arterial blood past the proximal edge of cuff 2. To control the distance of penetration, microcomputer 68 adjusts the reference pressure level communicated to pressure regulator 70 in response to distance estimates received from distance estimator 74 to maintain the estimated distance of penetration of arterial blood past the proximal edge of cuff 60 near the reference distance of penetration. For example, if the distance of penetration arterial blood past the proximal edge of cuff 2 becomes greater than the reference distance, microcomputer 68 acts to increase the reference pressure level, which causes more compression of limb 6 by cuff 2 thereby reducing the distance of penetration. If the distance of penetration arterial blood past the proximal edge of cuff 2 becomes less than the reference distance, microcomputer 68 decreases the reference pressure level causing less compression of limb 6 by cuff 2 thereby increasing the distance of penetration.

Microcomputer 68 monitors the cuff pressure level, cuff reference pressure level, and distance of penetration of arterial blood past the proximal edge of cuff 2. In response to any of these parameters exceeding predetermined alarm limits microcomputer 68 may alert a user to the presence of an alarm condition by activating alarm indicator 30.

Operating Room (OR) network interface 76 provides a means for microcomputer 68 to communicate with other instruments and data bases connected to an operating room information network. Microcomputer 68 may communicate cuff pressure levels, reference pressure levels, distances of penetration of arterial blood, reference distances, alarm conditions and other operating parameters of instrument 4. Microcomputer 68 may also receive information from other instruments such as patient monitoring equipment.

To enable a better understanding of the preferred embodiment, its typical use in a surgical procedure is described below.

A user first selects an appropriately sized cuff 2 for application to a portion of patient limb 6. Physiologic transducer 24 is then affixed to cuff 2 at transducer region 40 and cuff 2 is secured around the patient limb 6. Cuff 2 is applied to the limb such that transducer region 40 is proximal on the limb. Pneumatic passageways from instrument 4 to the inflatable portion of cuff 2 are completed by mating connectors 16 and 18, and connectors 12 and 14.

In response to user input via user interface 28 instrument 4 inflates cuff 2 to a level of pressure sufficient to stop blood flow past cuff 2. The level of pressure required in the inflatable portion of cuff 2 to stop blood flow past cuff 2 at a particular time is affected by many variables including the characteristics of cuff 2, the technique used in applying cuff 2, the physical characteristics of the portion of limb 6 to which cuff 2 is applied, and the physiological characteristics of the patient, including blood pressure.

Instrument 4 then estimates the distance of penetration of arterial blood past the proximal edge of cuff 2 and may operate to adjust the pressure level in the inflatable portion of cuff 2 to maintain the distance of penetration near a predetermined distance.

During the procedure, instrument 4 may communicate with a connected operating room information network via OR interface 76 as described above.

At the conclusion of the surgical procedure a user instructs instrument 4 to depressurize cuff 2 and cuff 2 is removed from the patient limb.

In the preferred embodiment described above a tourniquet cuff is the pressure applying apparatus that closes underlying arterial blood vessels in a portion of limb to stop blood flow. It will be apparent that alternate forms of the invention may use other types of pressure applying apparatus to selectively close arteries in other regions of the body where tissues may be compressed to stop arterial blood flow and that the invention may be adapted to sense and control the distance of penetration of blood in the arterial blood vessels beneath such other types of pressure applying apparatus. For example, to close an arterial vessel in an abdominal region of a patient, pressure-applying apparatus may be adapted to apply a controllable pressure to the surface of the abdomen at a desired location above the arterial vessel. By controlling the pressure applied by the apparatus, the invention can maintain the distance of penetration of blood in the artery beneath the pressure applying apparatus at a desired distance, thereby safely stopping the flow of arterial blood.

We claim:

1. Apparatus for estimating the distance of penetration of arterial blood into a portion of a patient limb encircled by a tourniquet cuff, comprising:
a cuff having proximal and distal side edges, configured for encircling a limb portion and for applying a pressure to the limb portion sufficient to stop arterial blood flow past the distal side edge;
physiologic transducer associated with the cuff and adapted for sensing a physiologic parameter indicative of penetration of arterial blood into the limb portion encircled by the cuff while blood flow past the limb portion is stopped; and
estimation means responsive to an output of the physiologic transducer for producing an estimate of the distance of penetration of arterial blood past the proximal edge of the cuff.

2. The apparatus as described in claim 1 wherein the cuff includes an inflatable portion having a length dimension between a first end and second end that is sufficient to encircle the limb portion to overlap the first end with the second end, thereby establishing an inflatable gas passageway around the limb portion, and wherein the physiologic transducer adjoins the inflatable gas passageway.

3. The apparatus as described in claim 2, wherein the physiologic transducer adjoins the inflatable portion at a predetermined location between the first and second ends.

4. The apparatus as described in claim 2, wherein the physiologic transducer is located adjacent to the first end of the inflatable portion, and wherein the inflatable portion further overlaps the physiologic transducer and the first end when the cuff encircles the limb portion.

5. The apparatus as described in claim 2 wherein the inflatable portion has an inner side adapted for facing the limb encircled by the cuff, and wherein the physiologic transducer is located between the inner side and the limb.

6. The apparatus as described in claim 2 wherein a physical property of the cuff near the location where the physiologic transducer adjoins the inflatable portion is configured to facilitate the sensing of the physiologic parameter by the physiologic transducer.

7. The apparatus as described in claim 6 wherein the physiologic transducer is an ultrasound transducer, and wherein the physical properties of the cuff near the location are predetermined to facilitate the passage of ultrasonic signals at the location.

8. The apparatus as described in claim 1 wherein the physiologic transducer is an ultrasound transducer and wherein the physiologic parameter is a diameter of the lumen of an artery within the limb portion encircled by the cuff; and
wherein the estimation means estimates the distance from the proximal side edge of the cuff to the most proximal location at which the diameter of the lumen is estimated to be zero.

9. The apparatus as described in claim 8 wherein the ultrasound transducer is a two-dimensional sensor array adapted for insonifying a three-dimensional volume of the limb portion.

10. The apparatus as described in claim 9 wherein the ultrasound transducer is comprised of a plurality of two-dimensional ultrasonic sensor arrays.

11. The apparatus as described in claim 1 wherein the physiologic transducer is comprised of a plurality of sensor elements adapted for sensing a plurality of physiologic parameters indicative of penetration of arterial blood into the limb portion.

12. The apparatus as described in claim 1 wherein the physiologic transducer is an arterial tonometer, wherein the physiologic parameter is pressure pulsation and wherein the estimation means estimates the distance between the proximal side edge of the cuff and the most proximal location at which the pressure pulsation is estimated to be zero.

13. The apparatus as described in claim 2 and further including a pressurizing means adapted for supplying pressurized gas to the inflatable portion of the cuff at a pressure sufficient to stop the flow of arterial blood past the limb portion.

14. The apparatus as described in claim 13 wherein the pressurizing means is responsive to the estimation means and adapted to regulate the pressure in the inflatable portion of the cuff so that the penetration of arterial blood into the limb portion is maintained near a predetermined distance relative to the proximal edge of the cuff.

15. The apparatus as described in claim 13 and further including control means for producing a signal indicative of a desired distance of penetration of arterial blood into the limb portion while blood flow past the cuff is stopped; and
wherein the pressurizing means is responsive to the control means and the estimation means to regulate the pressure in the inflatable portion of the cuff at a level that maintains the desired distance of penetration of arterial blood into the limb portion.

16. The apparatus as described in claim 1 and further including alarm means responsive to the estimation means for producing a signal perceptible to a user when the distance of penetration of arterial blood into the limb portion exceeds a predetermined distance limit threshold.

17. Apparatus for controlling the penetration of arterial blood beneath a device applying pressure to an arterial blood vessel, comprising:
pressure-application means for applying a controllable pressure to a body tissue located between the pressure application means and a selected arterial blood vessel;
penetration estimation means for estimating a distance of penetration of blood in the arterial blood vessel beneath the pressure application means; and
control means responsive to the estimation means for facilitating the control of the pressure applied to the body tissue by the pressure application means to stop the flow of blood in the vessel by maintaining the estimated distance of penetration near a selected penetration distance.

18. The apparatus as described in claim 17 wherein the pressure application means is a tourniquet cuff adapted for encircling a limb portion at a desired location, wherein the vessel is located in the body tissue encircled by the cuff, and wherein the control means is further adapted to stop the flow of blood in the vessel by maintaining the estimated distance of penetration beneath the tourniquet cuff near the selected penetration distance.

19. The apparatus as described in claim 17, wherein the pressure application means is further adapted for applying the controllable pressure to a predetermined area of body tissue at a selected abdominal location between the pressure application means and a selected arterial vessel, wherein the penetration estimation means further estimates the distance of penetration of blood in the arterial blood vessel beneath predetermined area of the pressure application means; and wherein the control means is further adapted to stop the flow of blood in the vessel by maintaining the estimated distance of penetration beneath the predetermined area of the pressure application means near the selected penetration distance.

20. Apparatus for controlling the lumen of an artery within a portion of a limb beneath a pressurizing cuff, comprising:
a pressurizing cuff for applying a pressure sufficient to close a lumen of an artery in a portion of a limb beneath the cuff;
lumen estimation means for estimating a location relative to the cuff at which the lumen is closed; and
control means responsive to the lumen estimation means for controlling the pressure applied by the cuff to maintain the location near a selected location.

21. A method for controlling the penetration of arterial blood beneath a device applying pressure to an arterial blood vessel, comprising the steps of:
applying a controllable pressure to a body tissue located between the pressure application means and a selected arterial blood vessel;
estimating a distance of penetration of blood in the arterial blood vessel beneath the pressure application means; and
controlling the pressure applied to the body tissue to stop the flow of blood in the vessel by maintaining the estimated distance of penetration near a selected penetration distance.

22. A method of controlling the lumen of an artery within a portion of a limb beneath a pressurizing cuff, comprising the steps of:
applying a pressure to a surface of a body that is sufficient to close a lumen of an artery in tissue beneath the surface of the body;
estimating a location at which the lumen is closed; and
controlling the pressure applied to the surface of the body to maintain the location at which the lumen is closed near a selected location.
